(19) United States
(12) Patent Application Publication
(10) Pub. No.: US 2012/0059252 A1
(43) Pub. Date: Mar. 8, 2012

(54) COMPUTER TOMOGRAPHY SORTING BASED ON INTERNAL ANATOMY OF PATIENTS

(75) Inventors: Ruijiang Li, Sunnyvale, CA (US); Steve B. Jiang, San Diego, CA (US); John Lewis, La Jolla, CA (US); Laura Cervino, La Jolla, CA (US)

(73) Assignee: THE REGENTS OF THE UNIVERSITY OF CALIFORNIA, Oakland, CA (US)

(21) Appl. No.: 13/320,237

(22) PCT Filed: May 13, 2010
(86) PCT No.: PCT/US10/34813

§ 371 Date: Nov. 11, 2011

Related U.S. Application Data
(60) Provisional application No. 61/177,985, filed on May 13, 2009.

(51) Int. Cl. A61B 6/00

(52) U.S. Cl. 600/425

(57) ABSTRACT

Methods, systems, and apparatus, including computer programs encoded on a computer storage medium, for computer tomography (CT) sorting based on internal anatomy of patients. CT scans of anatomical features of a human are obtained as pixels. From the scans, multiple respiratory features are determined. An optimal respiratory feature is selected and a respiratory signal is generated based on the multiple CT scans.

### CROSS-REFERENCE TO RELATED APPLICATIONS

This application claims priority to U.S. application Ser. No. 61/177,985 filed on May 13, 2009, and entitled “Four-dimensional computer tomography sorting based on internal anatomy of patients,” the entire contents of which are incorporated herein by reference.

### TECHNICAL FIELD

This specification relates to obtaining a computer tomography sorting algorithm based on patient internal anatomy, for example, by combining multiple respiratory features.

### BACKGROUND

Target definition is one of the steps in treatment planning for radiotherapy. The success of the treatment depends on the accuracy of the delineation of the target and organs at risk. Accurate target definition can be affected by target motion, due to, for example, patient respiration, which may cause significant motion artifacts in conventional free breathing computed tomography (CT) scans as commonly used for treatment planning. Four-dimensional computed tomography (4D CT) technique has been developed for the delineation of a moving target as well as target motion modeling.

4D CT can be accomplished by over-sampling CT data acquisition at each slice and sorting the images into multiple CT volumes corresponding to different respiratory states. There are two methods for acquiring 4D CT: cine mode and helical mode. For cine mode, the CT scanner continuously scans the patient at one couch position for a certain period of time (called the cine duration). Then, the X-ray beam is automatically turned off and the table is moved to the next position. The CT scanner begins another round of continuous scan at the new couch position. This process is repeated until a predetermined portion the body is fully covered. In the helical mode, the couch moves continuously at a very low speed while the X-ray source rotates around the patient.

### SUMMARY

This specification describes CT sorting based on internal anatomy of patients.

In general, one innovative aspect of the subject matter described here can be implemented as a computer-implemented method for generating a respiratory signal from computer tomography (CT) scans. Multiple CT scans of an anatomical feature of a human are retrieved by one or more data processing apparatuses from one or more computer-readable storage devices in which the multiple CT scans are stored. From the multiple CT scans, multiple respiratory features are determined. A respiratory signal for each respiratory feature is generated by the one or more data processing apparatuses directly from the multiple CT scans based on the multiple respiratory features and an optimal respiratory feature selected from the multiple respiratory features. From the respiratory signals identified for the multiple respiratory features, a spatial coherence is derived. The spatial coherence is an average pair-wise correlation coefficient. The correlation coefficient is a measure of a quality of the respiratory feature. The spatial coherence is calculated as

$$\frac{1}{{N}^{2}} {\sum }_{i = 1}^{N} {\sum }_{j = 1}^{N} \frac{{\sum }_{t = 1}^{T} ( {s}_{t}^{i} - {\overline{s}}^{i} ) ( {s}_{t}^{j} - {\overline{s}}^{j} )}{\sqrt{{( {s}_{t}^{i} - {\overline{s}}^{i} )}^{2} {( {s}_{t}^{j} - {\overline{s}}^{j} )}^{2}}} ,$$

wherein sⁱₜ is the respiratory signal at the ith slice position and at a particular couch position, N is a number of slice positions per couch position, T is a number of reconstructed axial CT slices per slice location, and

$${\overline{s}}^{i} = \frac{1}{T} {\sum }_{t = 1}^{T} {s}_{t}^{i}$$

is an average of sⁱₜ over time.

Another innovative aspect of the subject matter described here can be implemented as a computer-implemented method for generating a respiratory signal from CT scans. Multiple CT scans of an anatomical feature are retrieved via one or more data processing apparatuses from one or more computer-readable storage devices in which the multiple CT scans are stored. From the multiple CT scans, multiple respiratory features are determined by the one or more data processing apparatuses. A respiratory signal is generated directly from the multiple CT scans based on the multiple respiratory features and an optimal respiratory feature selected from the multiple respiratory features.

This, and other aspects, can include one or more of the following features the optimal respiratory feature can be selected from the multiple respiratory features. For each respiratory feature, CT scans used to determine the respiratory feature can be the seat from the one or more computer-readable storage devices, and respiratory signals generated based on the received CT scans are identified. From the respiratory signals identified for the multiple respiratory features, a spatial coherence, which is an average pair-wise correlation coefficient, is derived. The correlation coefficient is a measure of a quality of the respiratory feature. The spatial coherence can be determined from respiratory signals obtained from multiple slice positions per couch position and a number of reconstructed axis CT slices per slice location, and an average of respiratory signals over time. CT scans can be captured at a couch position which is one of a region around the upper thorax or a region below the diaphragm. The respiratory signal can be further processed to improve sorting accuracy. The processing can include applying a non-causal low pass filter to the respiratory signal and applying a cubic interpolation to obtain a smooth curve as a final respiratory signal. A CT scan can be stored in the one or more computer-readable storage devices as multiple pixels. A respiratory feature can be one or more of an air content, a lung area, a lung density, or a human body area. The body area can be the total number of pixels within a contour of the anatomical feature. The lung can be defined as a threshold of −350 Hounsfield Units (HU) plus a morphological smoothing operation. The lung area can be a total number of pixels within the lung. The lung density can be an average of CT numbers within the lung. Air content can be a summation of all CT numbers within the lung. Determining a respiratory feature can include identifying a contour of a polity of the human in a CT scan. The contour of the body can be scanned at a couch position that has a couch height. Identifying the contour of the body in the CT scan can include setting and image intensity measured in HU posterior to the couch height to a value, applying a threshold value measured in HU to find a body boundary, and using a morphological hole-filling operation to identified the body contour in each CT scan. The image intensity of posterior to the couch height can be set to −1000 HU. The threshold value can be set to −400 HU.

Yet another innovative aspect of the subject matter described here can be implemented as a computer-readable medium tangibly storing computer software instructions executable by data processing apparatus to perform operations for generating a respiratory signal from CT scans. The operations include processing multiple CT scans of an anatomical feature of a human to obtain multiple respiratory features, selecting an optimal respiratory feature from the multiple respiratory features, and generating a respiratory signal directly from the multiple CT scans based on the multiple respiratory features and an optimal respiratory feature selected based on the multiple respiratory features. The operations further include, for each respiratory feature, identifying respiratory signals generated based on CT scans used to determine the respiratory feature, and from the respiratory signals identified for the multiple respiratory features, the rising an average pair-wise correlation coefficient. The correlation coefficient can be a measure of a quality of the respiratory feature. The average pair-wise correlation coefficient can be determined from respiratory signals obtained from multiple slice positions per couch position and a number of reconstructed axis CT slices per slice location, and an average of respiratory signals over time. The operations can further include processing the respiratory signal to improve sorting accuracy by applying a non-causal low pass filter to the respiratory signal and applying a cubic interpolation to obtain a smooth curve as a final respiratory signal. A respiratory feature can be one or more of an air content, a lung area, a lung density, or a human body area. The body area can be the total number of pixels within a contour of the anatomical feature. The lung can be defined as a threshold of −350 HU plus a morphological smoothing operation. The lung area can be a total number of pixels within the lung. The lung density can be an average of CT numbers within the lung. Air content can be a summation of all CD numbers within the lung. Determining a respiratory feature can include identifying a contour of a body of the human in a CT scan. The contour of the body can be scanned at a couch position that has a couch height. Identifying the contour of the body in the CT scan can include setting and image intensity measured in HU posterior to the couch height to a value of −1000 HU, applying a threshold value of −400 HU to find a body boundary, and using a morphological poll-filling operation to identified the body contour in each CT scan.

Other innovative aspects of the subject matter described here can be implemented as a computer program which makes a computer execute procedures described here. Yet other innovative aspects of the subject matter can be implemented as a system that includes means for performing the operations described here.

Particular embodiments of the subject matter described in this specification can be implemented so as to realize one or more of the following advantages. When compared with a real-time position management (RPM) system, patient studies showed that the respiratory signals generated from internal features are similar to RPM signals for patients with regular breathing patterns (average correlation with RPM signals at all couch positions is above 0.92 in 9 out of 10 patients). The studies also showed that the 4D CT scan as described below produced sorted images with fewer motion artifacts for patients who exhibited relatively irregular breathing patterns. Thus, the use of internal anatomy for 4D CT sorting in thoracic and abdominal cancers is not only feasible but also has potential benefits. The sorting results are similar to those obtained with RPM signals for regular breathing patterns. For irregular breathing patterns, the respiratory signals obtained from the techniques described below present fewer artifacts relative to the RPM signal. The proposed algorithm is simple and robust, which makes it amenable for clinical implementation.

Alternative measures for the quality of respiratory signals may be investigated in the temporal domain, for example, smoothness. In parallel, other useful respiratory features may be combined easily with existing ones for better accuracy and more robustness. The described 4D CT internal sorting method eliminates the need of externally recorded surrogates of respiratory motion. The techniques are automatic, accurate, robust, cost efficient, and yet simple, and therefore can be readily implemented in clinical settings.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1a shows the external surrogates (RPM) and the four internal signals for two different couch positions for patient 4 (the first couch position (slices 25 to 28) is near the upper thorax region; air content is selected).

FIG. 1b shows the external surrogates (RPM) and the four internal signals for two different couch positions for patient 4 (the second couch position (slices 125 to 128) is below the diaphragm; body area is selected).

FIG. 2a is a coronal view of the MIP CT of patient 4 (grid has a vertical spacing of 2 cm, i.e., 2 couch positions).

FIG. 2b is correlation coefficients between RPM and five internal measures: air content, body area, lung area, lung density, and combined for all couch positions.

FIG. 3 is the RPM signal at 27 couch positions for patient 1. Each couch position contains 4 slice positions and at each slice position there are 16 axial CT slices. The RPM signal recorded during couch transition is not included, so it is not continuous in time. The arrow indicates where a sudden change of breathing amplitude occurred.

FIG. 4 shows images in coronal view sorted using the RPM signals (left) and the proposed method (right) at mid inhalation for patient 1. The two arrows on the left indicate significant sorting errors at the top of diaphragm with RPM signals. Images sorted by the proposed method and RPM signals are similar at other phases.

FIG. 5 is an example of an environment to capture 4D CT images.

FIG. 6 is an example of a system to process captured 4D CT images.

FIG. 7 is a flowchart of an example process to capture and process 4D CT images.

Like reference numbers and designations in the various drawings indicate like elements.

### DETAILED DESCRIPTION

The technology described relates to CT scans that account for respiratory motion during free breathing. In one example, a scanner, such as four-slice GE LightSpeed™ CT, acquires the CT data in cine mode. Respiratory signals can be optionally synchronously recorded for comparison purposes. To generate respiratory signals directly from the axial CT images, five respiratory features have been evaluated, namely, air content, body area, lung volume, conditional mean with threshold and conditional mean with percentile. For each four-slice couch position, a quantitative measure called spatial coherence was used to select the optimal internal features for generation of respiratory phases for slice sorting, which, along with the sorted CT images were compared with those obtained from a real-time position management (RPM) system, respectively.

Since the concept of 4D CT was introduced, some methods have been proposed to extract respiratory signals that can be used for slice sorting. They can be broadly divided into two categories. One is to use external signals recorded by extra instruments beside the CT scanner. Several types of instruments have been used to generate an external respiratory signal: reflective markers placed on the patient's body surface, that track the abdominal displacement, spirometers to measure the tidal volume through the mouth, and elastic belts to measure the pressure around the body. However, respiratory signals obtained from these external surrogates may not always accurately represent the internal target motion, especially when irregular breathing patterns occur. Using external surrogates that are poorly correlated with the actual internal target motion may cause severe organ mismatches or discontinuities in the retrospectively sorted CT volumes.

Another category of methods is based on extracting the respiratory signals from the axial CT images. There are a few methods proposed in the literature. One of the early works derived the respiratory signals by summing up the CT numbers in a given region of interest (ROI) and realigning the images based on the correlation between two adjacent locations in consecutive respiratory cycles. This method was demonstrated to work well around the diaphragm region. One drawback of the method is that the user has to specify an ROI for each couch position and may not work well for upper thoracic regions. In a related, yet more sophisticated method called internal air content analysis, the air-containing tissues are delineated using segmentation and the CT numbers within these tissues are essentially summed up to represent the internal air content in a particular CT slice or volume.

In the technology described here, air content was not used for sorting purposes but merely as a surrogate for internal motion to verify the correlation with the external spirometry measures. Recently, 4D CT sorting based on image registration or manifold learning techniques have been proposed. It has been suggested that these methods may result in smoother transitions between respiratory volumes at different phases. However, registration based methods require either a respiratory signal or a reference volume taken at breath-hold. Also, their computational burden and requirement for user supervision make them complicated for clinical implementation.

The usefulness of normalized cross correlation (NCC) for matching the respiratory phase of the CT images has been shown. More recently, a 4D CT sorting method based on NCC using an overlapping cine scan protocol has been proposed. The algorithm requires overlapping images from adjacent scan positions to perform sorting, leading to increased patient dose (approximately 33% increase for four-slice CT scan). There has also been some work on acquiring 4D cone-beam CT using internal anatomical features. However, most of these methods utilize features extracted from the diaphragm region which is not always present in the axial slices obtained from conventional fan-beam CT scans and therefore they are not applicable for the current study. Each of these sorting methods has its respective advantages and disadvantages depending on the anatomical features they use. However, there is no consensus on which is most suitable for 4D multi-slice CT sorting.

Respiratory motion during free breathing computed tomography (CT) scan may cause significant errors in target definition for tumors in thorax and upper abdomen. Four-dimensional (4D) CT technique has been widely used for treatment simulation of thoracic and abdominal cancer radiotherapy. In some 4D CT techniques, reconstructed CT slices over-sampled at the same couch position are retrospectively sorted. Some sorting methods depend on external surrogates of respiratory motion recorded by extra instruments. However, respiratory signals obtained from these external surrogates may not always accurately represent the internal target motion, especially when irregular breathing patterns occur.

As described below, multiple internal respiratory features are investigated and effectively combined based on a measure called spatial coherence with the goal of developing a clinically useful and practical method for 4D CT sorting. There is an important advantage of working directly with the CT images over external surrogates which rely on extra instruments. It not only eliminates the adverse effects of instrument malfunction and other sources of error (for example, loose skin contact, drift), but also makes the CT acquisition process much simpler. Such a simple and robust internal 4D CT sorting method can save a lot of human efforts and instrument costs for the clinic.

Such a new sorting method based on multiple internal anatomical features for multi-slice CT scan acquired in the cine mode is described below. Various features, including air content, lung area, lung density, and body area, are analyzed in this study. A measure called spatial coherence is used to select the optimal internal feature at each couch position and to generate the respiratory signals for 4D CT sorting. Spatial coherence is defined for each feature at each couch position. Different internal features can be selected at different couch positions, and the final 4D CT reconstruction can be built based on a combination of internal respiratory features.

In one implementation, the proposed method has been evaluated for 10 cancer patients (8 with thoracic cancer and 2 with abdominal cancer). For 9 patients, the respiratory signals generated from the combined internal features are well correlated to those from external surrogates recorded by the real-time position management (RPM) system (average correlation: 0.95±0.02), which is better than any individual internal measures at 95% confidence level. For these 9 patients, the 4D CT images sorted by the combined internal features are almost identical to those sorted by the RPM signal. For one patient with irregular breathing pattern, the respiratory signals given by the combined internal features do not correlate well with those from RPM (correlation: 0.68±0.42). In this case, the 4D CT image sorted by the method described presents fewer artifacts than that from the RPM signal.

In some implementations, a four-slice GE LightSpeed CT scanner (GE Medical Systems, Milwaukee, Wis., USA) is used to acquire the CT data for treatment simulation. The scanner is operated in the axial cine mode. The gantry speed was set to be 1 second per rotation. The cine duration was set to be the average observed breathing period of the patient plus an additional second to account for variations in breathing period. Depending on the respiratory period for each patient, a different number of axial CT slices (usually ranging from 11 to 20) were reconstructed from the X-ray projections at each couch position. Each CT slice has a thickness of 2.5 mm, so each scan at one couch position covers 1 cm of the patient body in the superior-inferior (SI) direction.

During the scan, a respiratory signal can be synchronously recorded by a Varian real-time position management (RPM) system (Varian Medical Systems, Inc, Palo Alto, Calif., USA), which tracked the motion of a reflective marker placed on the patient's abdomen. Note that the RPM signals are not needed for the internal sorting method. Rather, the RPM signals enable comparing with the estimated internal respiratory signals. CT data were collected from 10 cancer patients, out of whom 8 are thoracic cancer patients and 2 are abdominal cancer patients. By looking at the RPM signals, all the patients have regular breathing patterns except one thoracic cancer patient.

All the internal features require the identification of the patient body contour in each axial CT slice. First, the image intensities posterior to the couch height are set to −1000 Hounsfield units (HU). A threshold of −400 HU is then applied to find the body boundary and a morphological hole-filling operation is used to identify the body contour in each CT slice. In one implementation, the following four internal respiratory measures for each CT slice are used: body area, lung area, air content, and lung density. Their detailed definitions are as follows. Body area is the total number of pixels within the body contour. The lung is defined as a threshold of −350 HU plus a morphological smoothing operation. Lung area is the total number of pixels within the lung. Lung density is the average CT numbers within the lung. Air content is essentially the summation of all the CT numbers within the lung. The commonly used chest height is not used in this work since it is a less sensitive measure compared to body area and lung area.

The rationale of using body and lung areas as respiratory measures is based on the simple fact that both chest and lung expand during inhale and contract during exhale. They are, therefore, geometry-based measures. Note that body area is not a real internal anatomical measure. Here, the term “internal” is relative to the external surrogate recorded by external instruments. Lung density and air content are calculated from CT numbers and thus they are content-based measures. The reason why lung density is used is that when patients inhale, more air comes into the lung and this will decrease the average CT numbers. Notably, the air content is the product of the lung area and lung density. None of these four internal measures are equivalent measures and it is important to keep all of them for our sorting purposes.

Each of these internal measures has its respective pros and cons. For instance, air content and lung area are direct measures of internal anatomical change, but they may suffer from the interference of soft tissue (including heart, arteries and tumors) motion since these changes are not consistent with respiration. Lung density is less affected by these interferences but may not be robust. None of the above four measures work below the diaphragm since there is no meaningful air content or lung area. Body area does not suffer from the interference of soft tissue motion and works for the abdominal region, however, depending on the way the patient breathes (chest versus abdomen), it may not change much during a breathing cycle. In light of this, it may be beneficial to combine these measures to generate more robust respiratory signals.

For multi-slice CT scans, an ideal respiratory signal should be exactly the same for all the slices at the same couch position since they are acquired at the same time. Therefore, a necessary condition for good internal features is that they should produce similar respiratory signals for all the slices at the same couch position. The quality of a given internal feature can be measured by spatial coherence, which is defined as the average pair-wise correlation coefficient among all the respiratory signals derived from this feature at the same couch position. Denoting sⁱₜ as the respiratory signal at the ith slice position and at a particular couch position, the spatial coherence is calculated as:

$$\frac{1}{{N}^{2}} {\sum }_{i = 1}^{N} {\sum }_{j = 1}^{N} \frac{{\sum }_{t = 1}^{T} ( {s}_{t}^{i} - {\overline{s}}^{i} ) ( {s}_{t}^{j} - {\overline{s}}^{j} )}{\sqrt{{( {s}_{t}^{i} - {\overline{s}}^{i} )}^{2} {( {s}_{t}^{j} - {\overline{s}}^{j} )}^{2}}}$$

In the above equation, N is the number of slice positions per couch position (N=4, for example), T is the number of reconstructed axial CT slices per slice location (T varies between 11 and 20 for different patients, for example), and

$${\overline{s}}^{i} = \frac{1}{T} {\sum }_{t = 1}^{T} {s}_{t}^{i}$$

is the average of sⁱₜ over time. The feature that has the largest spatial coherence is selected and the corresponding respiratory signals for the four slices are averaged to generate the final signal for that couch position. Thus, spatial coherence is an average correlation coefficient that is determined from respiratory signals obtained from multiple slice positions per couch position and a number of reconstructed axis CT slices per slice location. The spatial coherence is also a function of an average of respiratory signals over time.

The spatial coherence is defined for each feature at each couch position. Different internal features can be selected at different couch positions, and therefore the final 4D CT reconstruction can be built based on a combination of internal respiratory features. For example, the air content measure is favored because it incorporates both lung area and lung density information and in general produces better sorting results than other features even at a similar level of spatial coherence. Thus, air-content measure is selected whenever its spatial coherence is above a certain threshold for a particular couch position; otherwise, the spatial feature with the highest spatial coherence is selected. In some implementations, a threshold value of 0.98 can be selected for selecting the air content based on the evaluation results. Alternatively, other threshold values can also be used. The respiratory signals corresponding to the selected feature are averaged for the four slices to generate the final signal for that couch position.

Since all patients except one have regular breathing patterns, the quality of the respiratory signals obtained from each individual measure and from the combined measures is judged by correlating with the RPM signal at all couch positions. Notice that both lung density and air content are negative due to negative CT numbers. Consequently, all their values need to be changed to positive ones in order to be consistent with RPM signals (i.e., peak corresponds to inhale; valley corresponds to exhale). The original values were used for both body area and lung area, since they are already consistent with RPM signals. The effectiveness of our combined signal was evaluated by comparing its correlation with RPM signal with the correlation between each individual measure and RPM signals through a one-side paired t-test among all the patients. If a high correlation is obtained between the combined signal and RPM signal, it can be expected that the corresponding sorted CT images will be similar too. On the other hand, if the correlation is not sufficiently high, noticeable differences between the sorted CT images are expected. All the 4D CT images sorted by the new method as well as by the RPM signals are qualitatively compared by visual inspection.

Unlike the typically smooth RPM signal sampled at 30 Hz, internal respiratory signals can be obtained at specified time instances (usually 11 to 20 time points during 1 breathing cycle). They may also exhibit noisy fluctuations depending on the underlying features they use. Post-processing of the respiratory signals may help improve the sorting accuracy. In some implementations, a non-causal low pass filter can be applied to the original respiratory signals. The impulse response of the filter can be [0.6 1 0.6], leading to a normalized cut-off frequency of about 0.3. This is basically a weighted average of the current signal and its two neighboring points for every time instance. Then a cubic interpolation can be performed to obtain a smooth curve as our final respiratory signal. Phase-based sorting was performed to obtain 10 CT volumes, corresponding to those obtained with RPM signals.

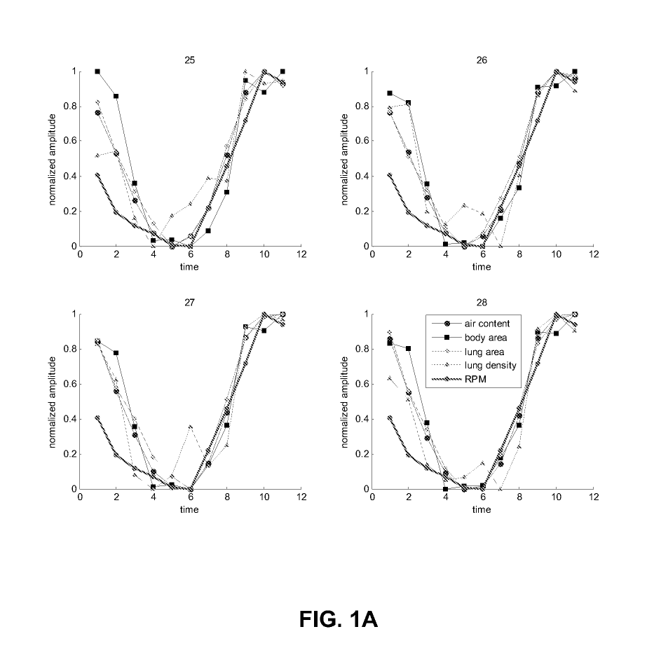

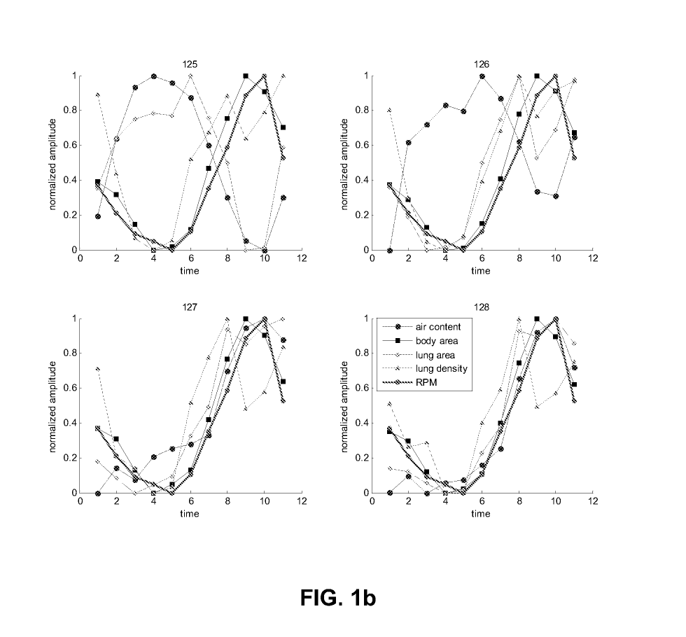

FIG. 1a shows the four internal and the combined measures as well as the external surrogates (RPM) for two different couch positions for patient 4. Note that all signals are normalized between 0 and 1 for illustration purposes. The first couch position is around the upper thorax region. For this couch position, the air content measure was selected with the highest spatial coherence of 0.995, leading to a correlation of 0.92 with RPM. Both lung area and body area also work well for this couch position (correlation 0.92 and 0.89 with RPM, respectively). The lung density measure is more erratic and does not correlate well with RPM (correlation 0.81).

FIG. 1b illustrates the second couch position which is right below the diaphragm region. For this couch position, the body area measure was selected, with the highest spatial coherence of 0.998, leading to a correlation of 0.98 with RPM. None of the other four measures works well for this couch position. The air content and lung area yield spatial coherences of −0.05 and 0.19, correlation of −0.03 and 0.65 with RPM, respectively because there is no meaningful lung region at this couch position.

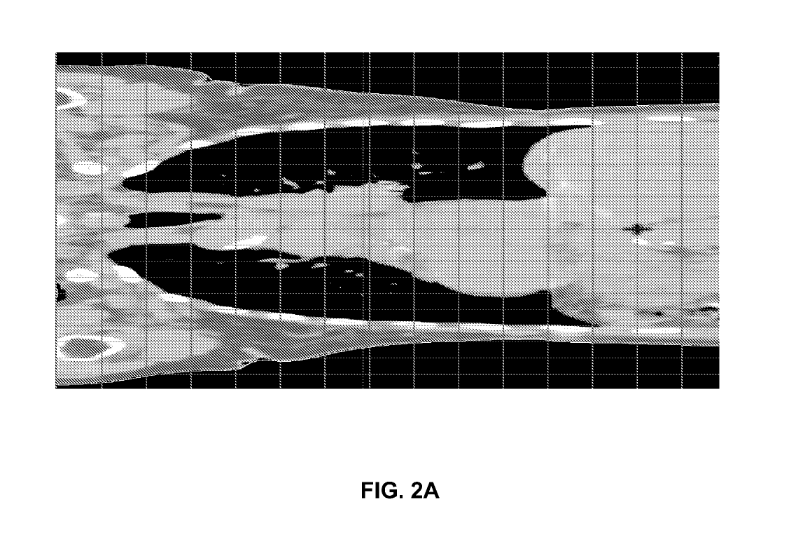

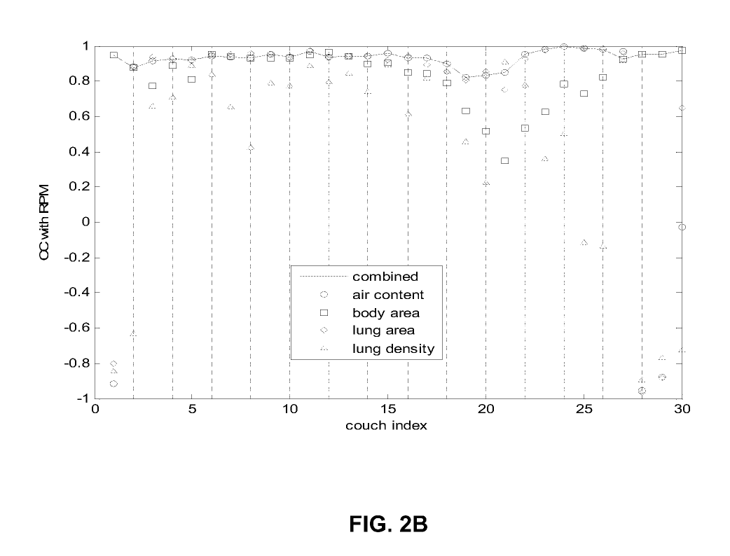

FIG. 2a shows the correlation coefficients between RPM and the four internal and combined measures for all couch positions for patient 4. For illustration purpose, the coronal view of the maximum intensity projection (MIP) of RPM sorted 4D CT is also shown, which is aligned with the couch index in FIG. 2(b). Air content and lung area work for the upper thorax region but not near the abdominal region. Lung density is in general less robust than the other three internal measures. Body area gives erratic signals for the lower part of the lung for patient 4 but works for both upper thoracic and upper abdominal regions. Overall, the proposed method selected the internal measures with highest correlation with RPM for most couch positions. At those couch positions where the best internal measures were not selected, the correlation of the selected measure with RPM is already high and very close to the best one.

By combining the four measures using spatial coherence, a respiratory signal better correlated with RPM signals in general was achieved. Table 1 lists the correlation between the 4 internal and combined measures and RPM signals for all the 10 patients.

TABLE 1
Correlation with RPM signals for the 4 internal and combined measures for 10
patients (patients 1 to 8 are thoracic cancer patients and patients 9 and 10 are
abdominal cancer patients)

| Method/ | | | | | |
|---|---|---|---|---|---|
| CC with | Air | | | Lung | |
| RPM | content | Body area | Lung area | density | Combined |
| Patient 1 | 0.66 ± 0.43 | 0.39 ± 0.47 | 0.61 ± 0.44 | 0.08 ± 0.40 | 0.68 ± 0.42 |
| Patient 2 | 0.82 ± 0.49 | 0.93 ± 0.07 | 0.81 ± 0.46 | 0.67 ± 0.51 | 0.97 ± 0.03 |
| Patient 3 | 0.67 ± 0.53 | 0.94 ± 0.03 | 0.72 ± 0.36 | 0.13 ± 0.70 | 0.93 ± 0.07 |
| Patient 4 | 0.71 ± 0.56 | 0.84 ± 0.15 | 0.73 ± 0.53 | 0.41 ± 0.59 | 0.93 ± 0.04 |
| Patient 5 | 0.75 ± 0.48 | 0.92 ± 0.06 | 0.72 ± 0.48 | 0.55 ± 0.56 | 0.94 ± 0.07 |
| Patient 6 | 0.75 ± 0.60 | 0.69 ± 0.27 | 0.77 ± 0.55 | 0.68 ± 0.63 | 0.98 ± 0.02 |
| Patient 7 | 0.85 ± 0.26 | 0.92 ± 0.05 | 0.84 ± 0.24 | 0.51 ± 0.58 | 0.93 ± 0.06 |
| Patient 8 | 0.56 ± 0.72 | 0.85 ± 0.21 | 0.54 ± 0.70 | 0.35 ± 0.73 | 0.96 ± 0.08 |
| Patient 9 | 0.53 ± 0.72 | 0.97 ± 0.04 | 0.55 ± 0.66 | −0.04 ± 0.79 | 0.98 ± 0.04 |
| Patient 10 | 0.34 ± 0.79 | 0.71 ± 0.46 | 0.37 ± 0.72 | −0.02 ± 0.54 | 0.95 ± 0.06 |
| Grand | 0.66 ± 0.16 | 0.82 ± 0.18 | 0.67 ± 0.14 | 0.33 ± 0.28 | 0.92 ± 0.09 |
| mean ± std | | | | | |
| p values | <0.001 | 0.01 | <0.001 | <0.001 | — |

Except for patient 1, the average correlations with RPM are all above 0.93, with a standard deviation less than or equal to 0.08 over all the couch positions. If patient 1 is excluded, the average correlation with RPM would be around 0.95, with a standard deviation of around 0.02. This suggests that for regular breathing patterns, the combined internal measures may be treated as a good approximation for externally measured RPM signals based on this limited data set. Through visual inspection, it was found that for these 9 patients, the new sorting method and the RPM method generate sorted 4D CT images with negligible difference. It was also observed that except for patient 3, where the body area gives slightly higher correlation (the reason why the combined measure is slightly worse than body area for patient 3 is because a slightly more weight was given to air content over body area), the combined measures outperform any of the 4 internal measures in terms of average correlation with RPM, demonstrating the effectiveness of our combination algorithm. To be sure, a one-sided paired t-test was performed on the correlation with RPM between the combined measure and each of the internal measures. The p-values are listed in Table 1. Results suggest that at a confidence level of 95%, the combined method described above is better correlated with RPM than any of the 4 individual internal measures.

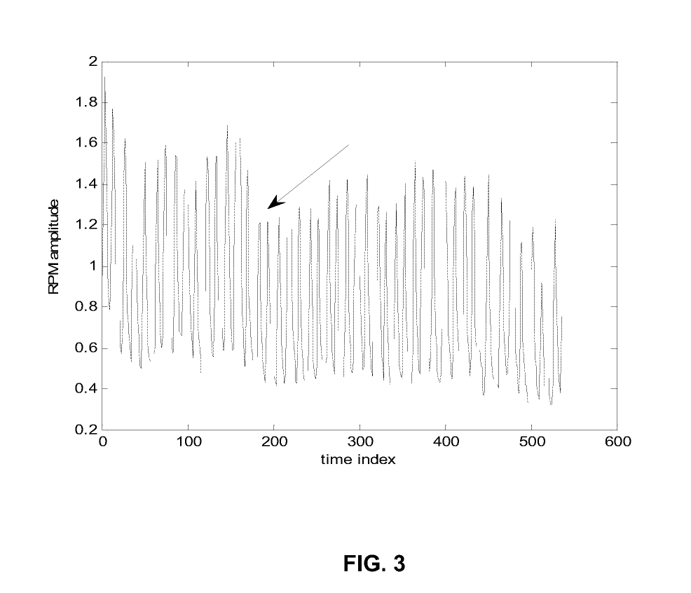

Patient 1 exhibited relatively irregular breathing patterns during the CT scan. FIG. 3 shows the RPM amplitude at all the 27 couch positions for patient 1. There is a sudden change in the range of breathing amplitude around 8^{th} couch position, indicated by an arrow in FIG. 3. Although the average correlation between RPM and combined internal signals is only around 0.68 for this patient, there does not seem to be significant differences between the two sorted images for all the 10 phases except at mid inhalation.

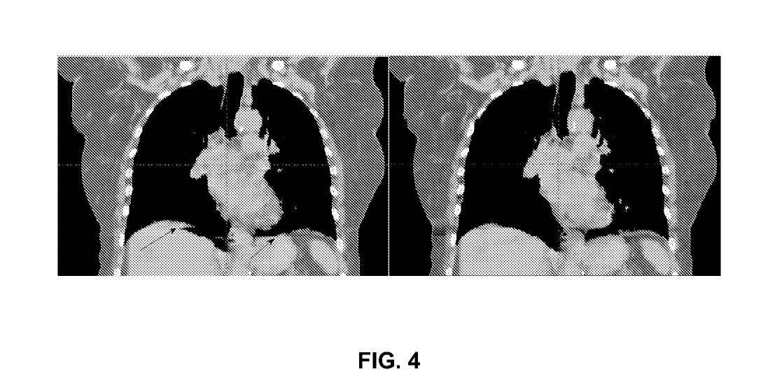

FIG. 4 shows the images in coronal view sorted using both the proposed method and RPM signals at mid inhalation for patient 1. Note the sorting errors at the top of diaphragm with RPM signals.

In some implementations, the sorting algorithm can be implemented on the MatLab 7.7 platform. With an Intel Core 2 Quad 2.67 GHz CPU and an 8 GB RAM, it takes about 5 to 10 minutes to sort a 4D CT data set using our method, depending upon the total number of CT images acquired over all the couch positions. Note that prior methods take about 30 minutes to sort a 4D CT data set. While these computation times are not negligible even during the treatment planning stage, a feature of the method described here is that each couch position is completely independent, thus making it possible to benefit from a parallel computing environment. Computation time can also be reduced by implementing and optimizing the code in a low-level language. Note that the internal signals described here are local measures in contrast to external surrogates. Regardless, the internal signals described here are more accurate than phase-based sorting.

It is conceivable that there may be phase shift between motion at different parts of the thorax or abdomen. For instance, peak inhale at upper thorax might correspond to mid inhale at lower thorax for some patients. The sorting method based on internal measures puts those CT slices with peaks of breathing signals at all couch positions into the same CT volume, which means that peak inhale at both upper and lower thorax will be incorrectly put into the same CT volume. The resulting volume does not correspond to the actual patient geometry. It was observed that for the 9 patients with regular breathing patterns in this study, the phase shift between RPM and internal signal is within 0.4 seconds in almost all cases. This is in agreement with previous studies, where a maximum of 0.4 seconds shift between diaphragm and tumor motion was found among 10 lung cancer patients using fluoroscopic images. Therefore, it is expected that the effect of the space-dependent phase shift on the accuracy internal sorting will be minimal.

Another study found a phase shift of −0.65 to 0.5 second between tumor motion and external surrogate on an intra-fractional scale. This is different from space-dependent phase shift. It is time-dependent and creates problems for external sorting. However, this shift alone does not affect internal sorting. If, for instance, a patient breathes regularly without space-dependent phase shift, and the peak of the external surrogate corresponds to that of the breathing, then, during CT scan, this correspondence may become invalid as time goes by (for example, peak of the external surrogate may correspond to the mid inhale or exhale). External sorting will incorrectly put those CT slices at the peaks of the external surrogate into the same CT volume, which now corresponds to different breathing phases. But internal measures are always consistent with breathing in this case. In reality, the phase shift is most likely to be a mixture of these two different kinds of phase shift. Based on the 4D CT images for the 10 patients we studied, it seems that the impact of these two phase shift effects on 4D CT quality is minimal.

In summary, a new 4D CT sorting method based on multiple internal anatomical features is described. A measure called spatial coherence is used to combine them and generate the final respiratory signal. The described method eliminates the need for externally recorded surrogates of respiratory motion. Patient studies showed that the respiratory signals generated from internal features are similar to RPM signals for regular breathing patterns and result in better sorted images for relatively irregular breathing patterns. The main advantage of the proposed method is its simplicity and robustness, relative to the individual internal measures, and thus it is amenable for clinical implementation.

The aforementioned techniques for obtaining respiratory signals from CT scans of anatomical features can be implemented as computer software instructions executable by a data processing apparatus. Alternatively, or in addition, the techniques can be implemented in a system that includes data processing apparatus and a computer-readable medium that encodes computer software instructions executable by the data processing apparatus.

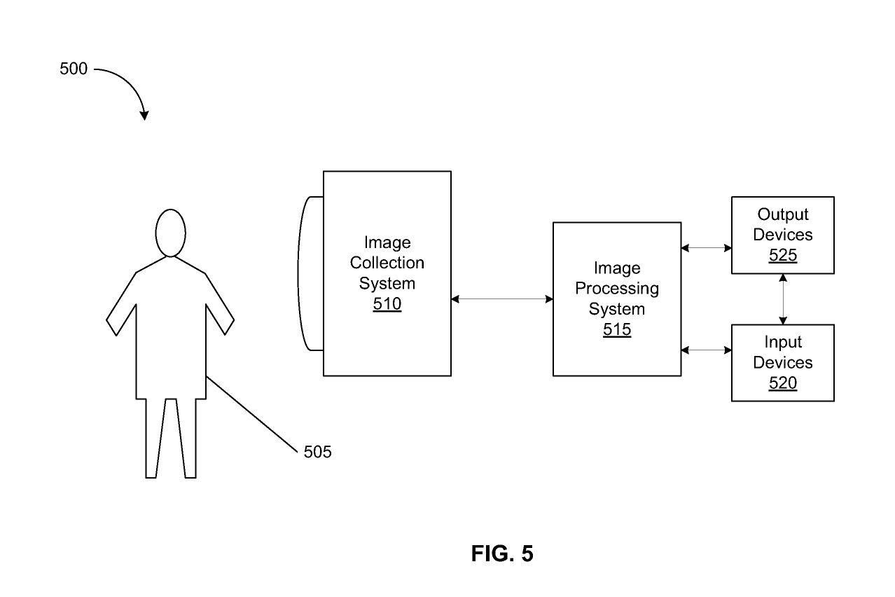

FIG. 5 is an example of an environment 500 to capture 4D CT images for generating a respiratory signal. An image collection system 510 scans a human patient 505 to obtain CT scans. A four-slice GE LightSpeed CT scanner (GE Medical Systems, Milwaukee, Wis., USA) is an example of an image collection system 510 used to obtain scan data. The image collection system 510 can be operated in an axial cine mode to obtain the multiple CT scans. A cine duration of the image collection system 505 can be set to be an average observed breathing period of the human plus an additional second to account for variations in breathing period. A gantry speed of the image collection system 510 can be set to be 1 second per rotation. Each CT scan can have a thickness of 2.5 mm such that each scan at a couch position covers 1 cm of the body of the human in the superior-inferior (SI) direction.

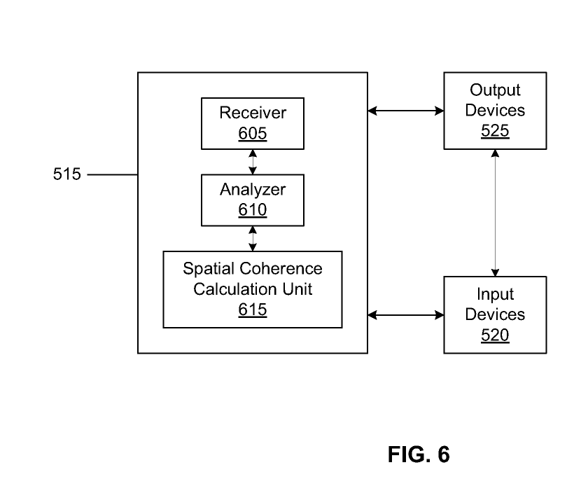

An image processing system 520 is operatively coupled to the image collection system 510 and is configured to receive the CT scan data from the image collection system 510. The image processing system 510, in some implementations, can include a data processing apparatus configured to execute computer software instructions. For example, the data processing apparatus can execute computer software instructions to receive the CT scan data from the image collection system 510. Input devices 520 and output devices 525 can be operatively coupled to the image processing system 515 and to each other. The input devices 520 can include a keyboard, a mouse, a keypad, a touch screen, and the like. The output devices 525 can include a computer monitor and the like. Upon receiving the CT scan data from the image collection system 510, the image processing system 515 is configured to perform operations described with reference to FIG. 6.

FIG. 6 is an example of a system 515 to process captured 4D CT images. In some implementations, the system 515 can include a receiver 605 configured to obtain multiple computer tomography (CT) scans of an anatomical feature of a human. Each CT scan can include multiple pixels. In some implementations, the system 515 can include a data storage device to store the multiple CT scans. The image capturing unit 510 can capture the CT scans at a couch position which is one of a region around the upper thorax or a region below the diaphragm. A number of CT scans obtained at each couch position can range from eleven to twenty.

In some implementations, the system 515 can include an analyzer 610 to analyze the CT scans to obtain the respiratory signals as described above. The analyzer 610 is an example of data processing apparatus configured to execute computer software instructions. As described previously, a respiratory feature is one or more of an air content, a lung area, a lung density, or a human body area. The body area is the total number of pixels within a contour of the anatomical feature. The lung is a threshold of −350 Hounsfield Units (HU) plus a morphological smoothing operation. The lung area is a total number of pixels within the lung. The lung density is an average of CT numbers within the lung. Air content is a summation of all CT numbers within the lung.

In some implementations, the analyzer 610 can be configured to determine a respiratory feature by identifying a contour of a body of the human in a CT scan. The CT scan can be an axial CT scan. The contour of the body can be scanned at a couch position that has a couch height. To identify the contour of the body in the CT scan, the analyzer 610 can set an image intensity measured in Hounsfield units (HU) posterior to the couch height to a value, apply a threshold value measured in HU to find a body boundary, and use a morphological hole-filling operation to identify the body contour in each CT scan. The image intensity posterior to the couch height can be set to −1000 HU. The threshold value can be set to −400 HU.

In some implementations, the analyzer 610 can further process the respiratory signal to improve sorting accuracy. To do so, the analyzer 610 can apply a non-causal low pass filter to the respiratory signal and apply a cubic interpolation to obtain a smooth curve as a final respiratory signal. Furthermore, the analyzer 61000 can sort the multiple CT scans based on the selected optimal respiratory feature.

The system 515 can further include a spatial coherence calculation unit 615 configured to determine a spatial coherence. For example, for each respiratory feature, the spatial coherence calculator unit 615 can identify CT scans from which the respiratory feature was identified, identify respiratory signals generated based on the identified CT scans, and derive, from the identified respiratory signals, a spatial coherence which is an average pair-wise correlation coefficient. As described previously, the spatial coherence is a correlation coefficient and a measure of a quality of the respiratory feature. Based on the spatial coherence, the image processing system 515 can select an optimal respiratory feature from the multiple respiratory features, and generate a respiratory signal directly from the multiple CT scans based on the multiple respiratory features. Furthermore, for each respiratory feature, the spatial coherence calculation unit 615 can be configured to select different anatomical features at different couch positions, define a spatial coherence for each selected different anatomical feature, build a final four dimensional CT reconstruction based on a combination of respiratory features, for each of which a corresponding spatial coherence is defined.

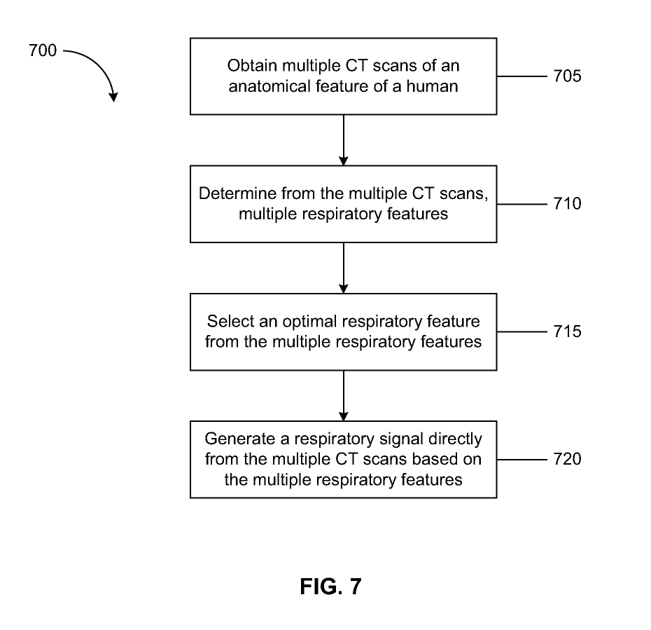

FIG. 7 is a flowchart of an example process 700 to capture and process 4D CT images. The process 700 can be implemented as computer software instructions executable by a data processing apparatus. The process 700 obtains multiple CT scans of an anatomical feature of a human (step 705). The process determines from the multiple CT scans, multiple respiratory features (step 710). The process selects an optimal respiratory feature from the multiple respiratory features (step 715). The process generates a respiratory signal directly from the multiple CT scans based on the multiple respiratory features (step 720).

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on a computer storage medium for execution by, or to control the operation of, data processing apparatus. Alternatively or in addition, the program instructions can be encoded on an artificially-generated propagated signal, for example, a machine-generated electrical, optical, or electromagnetic signal, that is generated to encode information for transmission to suitable receiver apparatus for execution by a data processing apparatus.

A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

In some implementations, a computer program can be stored in one or more computer-readable storage devices and can be executed by one or more data processing apparatuses. For example, a portion of the computer program can be stored on a computer-readable storage device whereas another portion of the computer program can be stored on another computer-readable storage device. Further, a data processing apparatus can retrieve a portion of the computer program from a computer-readable storage device and execute the retrieved portion to perform an operation or a procedure. Another data processing apparatus can retrieve another portion of the computer program from another computer-readable storage device and execute the other retrieved portion to perform another operation or another procedure. The combination of the procedure and the other procedure can form all or portions of the output. The system can operate based on an inter-relationship between the one or more computer-readable storage devices and the one or more data processing apparatuses.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry. To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

While this specification contains many specific implementation details, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the invention have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. For example, the spatial coherence used is only a necessary condition for an ideal respiratory signal. To get better signals, other constraints in the temporal domain can be applied, for example, smoothness (using quadratic variation) and temporal coherence at the same couch position. In some implementations, other meaningful internal respiratory measures can be investigated using advanced image processing techniques. These additional measures can be easily combined with existing ones. In order to compare our new sorting method with established methods based on external surrogates, a new quantitative and direct measure for the quality of 4D CT sorting can be used. Similarity measures among gross target volumes (GTV) for all the phases using deformable models can be explored. Also, the proposed method may be extended to the helical acquisition mode.

A few implementations have been described. Variations and enhancements of the described implementations and other implementations can be made based on what is described and illustrated.

What is claimed is:

1. A computer-implemented method for generating a respiratory signal from computer tomography (CT) scans, the method comprising:
retrieving, by one or more data processing apparatuses, a plurality of CT scans of an anatomical feature of a human from one or more computer-readable storage devices in which the plurality of CT scans are stored;
determining, by the one or more data processing apparatuses, from the plurality of CT scans, a plurality of respiratory features;
generating, by the one or more data processing apparatuses, a respiratory signal for each respiratory feature directly from the plurality of CT scans based on the plurality of respiratory features and an optimal respiratory feature selected from the plurality of respiratory features; and
from the respiratory signals identified for the plurality of respiratory features, deriving a spatial coherence which is an average pair-wise correlation coefficient, wherein the correlation coefficient is a measure of a quality of the respiratory feature, wherein the spatial coherence is calculated as
$$\frac{1}{{N}^{2}} {\sum }_{i = 1}^{N} {\sum }_{j = 1}^{N} \frac{{\sum }_{t = 1}^{T} ( {s}_{t}^{i} - {\overline{s}}^{i} ) ( {s}_{t}^{j} - {\overline{s}}^{j} )}{\sqrt{{( {s}_{t}^{i} - {\overline{s}}^{i} )}^{2} {( {s}_{t}^{j} - {\overline{s}}^{j} )}^{2}}} ,$$
wherein sⁱₜ is the respiratory signal at the ith slice position and at a particular couch position, N is a number of slice positions per couch position, T is a number of reconstructed axial CT slices per slice location, and
$${\overline{s}}^{i} = \frac{1}{T} {\sum }_{t = 1}^{T} {s}_{t}^{i}$$
is an average of sⁱₜ over time.

2. A computer-implemented method for generating a respiratory signal from computer tomography (CT) scans, the method comprising:
retrieving, by one or more data processing apparatuses, a plurality of CT scans of an anatomical feature of a human from one or more computer-readable storage devices in which the plurality of CT scans are stored;
determining, by the one or more data processing apparatuses, from the plurality of CT scans, a plurality of respiratory features; and
generating, by the one or more data processing apparatuses, a respiratory signal directly from the plurality of CT scans based on the plurality of respiratory features and an optimal respiratory feature selected from the plurality of respiratory features.

3. The method of claim 1, further comprising selecting, by the one or more data processing apparatuses, the optimal respiratory feature from the plurality of respiratory features.

4. The method of claim 1, further comprising:
for each respiratory feature:
receiving, from the one or more computer-readable storage devices, CT scans used to determine the respiratory feature,
identifying respiratory signals generated based on the received CT scans; and
from the respiratory signals identified for the plurality of respiratory features, deriving a spatial coherence which is an average pair-wise correlation coefficient, wherein the correlation coefficient is a measure of a quality of the respiratory feature.

5. The method of claim 4, wherein spatial coherence is calculated as
$$\frac{1}{{N}^{2}} {\sum }_{i = 1}^{N} {\sum }_{j = 1}^{N} \frac{{\sum }_{t = 1}^{T} ( {s}_{t}^{i} - {\overline{s}}^{i} ) ( {s}_{t}^{j} - {\overline{s}}^{j} )}{\sqrt{{( {s}_{t}^{i} - {\overline{s}}^{i} )}^{2} {( {s}_{t}^{j} - {\overline{s}}^{j} )}^{2}}} ,$$
wherein sⁱₜ is the respiratory signal at the ith slice position and at a particular couch position, N is a number of slice positions per couch position, T is a number of reconstructed axial CT slices per slice location, and
$${\overline{s}}^{i} = \frac{1}{T} {\sum }_{t = 1}^{T} {s}_{t}^{i}$$
is an average of sⁱₜ over time.

6. The method of claim 4, wherein the spatial coherence is determined from respiratory signals obtained from a plurality of slice positions per couch position and a number of reconstructed axis CT slices per slice location, and an average of respiratory signals over time.

7. The method of claim 1, wherein CT scans are captured at a couch position which is one of a region around the upper thorax or a region below the diaphragm.

8. The method of claim 1, further comprising processing the respiratory signal to improve sorting accuracy, the processing comprising applying a non-causal low pass filter to the respiratory signal and applying a cubic interpolation to obtain a smooth curve as a final respiratory signal.

9. The method of claim 1, wherein a CT scan is stored in the one or more computer-readable storage devices as a plurality of pixels, wherein a respiratory feature is one or more of an air content, a lung area, a lung density, or a human body area.

10. The method of claim 9, wherein the body area is the total number of pixels within a contour of the anatomical feature.

11. The method of claim 9, wherein the lung is defined as a threshold of −350 Hounsfield Units (HU) plus a morphological smoothing operation.

12. The method of claim 9, wherein the lung area is a total number of pixels within the lung.

13. The method of claim 9, wherein the lung density is an average of CT numbers within the lung.

14. The method of claim 13, wherein air content is a summation of all CT numbers within the lung.

15. The method of claim 1, wherein determining a respiratory feature comprises identifying a contour of a body of the human in a CT scan, wherein the contour of the body is scanned at a couch position that has a couch height, and wherein identifying the contour of the body in the CT scan comprises:
setting an image intensity measured in Hounsfield units (HU) posterior to the couch height to a value;
applying a threshold value measured in HU to find a body boundary; and
using a morphological hole-filling operation to identify the body contour in each CT scan.

16. The method of claim 15, wherein the image intensity posterior to the couch height is set to −1000 HU.

17. The method of claim 15, wherein the threshold value is set to −400 HU.

18. A computer-readable medium tangibly storing computer software instructions executable by data processing apparatus to perform operations for generating a respiratory signal from computer tomography (CT) scans, the operations comprising:
processing a plurality of CT scans of an anatomical feature of a human to obtain a plurality of respiratory features;
selecting an optimal respiratory feature from the plurality of respiratory features; and
generating a respiratory signal directly from the plurality of CT scans based on the plurality of respiratory features and an optimal respiratory feature selected based on the plurality of respiratory features.

19. The computer-readable medium of claim 18, the operations further comprising:
for each respiratory feature, identifying respiratory signals generated based on CT scans used to determine the respiratory feature; and
from the respiratory signals identified for the plurality of respiratory features, deriving an average pair-wise correlation coefficient, wherein the correlation coefficient is a measure of a quality of the respiratory feature.

20. The computer-readable medium of claim 19, wherein the average pair-wise correlation coefficient is determined from respiratory signals obtained from a plurality of slice positions per couch position and a number of reconstructed axis CT slices per slice location, and an average of respiratory signals over time.

21. The computer-readable medium of claim 18, the operations further comprising processing the respiratory signal to improve sorting accuracy by applying a non-causal low pass filter to the respiratory signal and applying a cubic interpolation to obtain a smooth curve as a final respiratory signal.

22. The computer-readable medium of claim 18, wherein a respiratory feature is one or more of an air content, a lung area, a lung density, or a human body area,
wherein the body area is the total number of pixels within a contour of the anatomical feature,
wherein the lung is defined as a threshold of −350 Hounsfield Units (HU) plus a morphological smoothing operation,
wherein the lung area is a total number of pixels within the lung,
wherein the lung density is an average of CT numbers within the lung, and
wherein air content is a summation of all CT numbers within the lung.

23. The computer-readable medium of claim 18, wherein determining a respiratory feature comprises identifying a contour of a body of the human in a CT scan, wherein the contour of the body is scanned at a couch position that has a couch height, and wherein identifying the contour of the body in the CT scan comprises:
setting an image intensity measured in Hounsfield units (HU) posterior to the couch height to a value of −1000 HU;
applying a threshold value of −400 HU to find a body boundary; and
using a morphological hole-filling operation to identify the body contour in each CT scan.

24. A program which makes a computer execute procedures, the procedures comprising:
receiving a plurality of computer tomography (CT) scans of an anatomical feature of a human, wherein each CT scan comprises a plurality of pixels;
determining from the plurality of CT scans, a plurality of respiratory features;
selecting, by the data processing apparatus, an optimal respiratory feature from the plurality of respiratory features; and
generating a respiratory signal directly from the plurality of CT scans based on the plurality of respiratory features.

25. The program of claim 24, the procedures further comprising, for each respiratory feature:
receiving CT scans from which the respiratory feature was determined, and
identifying respiratory signals generated based on the received CT scans; and from the respiratory signals identified for the plurality of respiratory features, deriving a spatial coherence which is an average pair-wise correlation coefficient, wherein the correlation coefficient is a measure of a quality of the respiratory feature.

26. The program of claim 24, the procedures further comprising processing the respiratory signal to improve sorting accuracy by applying a non-causal low pass filter to the respiratory signal and applying a cubic interpolation to obtain a smooth curve as a final respiratory signal.

27. The program of claim 24, wherein a respiratory feature is one or more of an air content, a lung area, a lung density, or a human body area,
wherein the body area is the total number of pixels within a contour of the anatomical feature,
wherein the lung is defined as a threshold of −350 Hounsfield Units (HU) plus a morphological smoothing operation,
wherein the lung area is a total number of pixels within the lung,
wherein the lung density is an average of CT numbers within the lung, and
wherein air content is a summation of all CT numbers within the lung.

28. A system comprising:
means for receiving a plurality of computer tomography (CT) scans of an anatomical feature of a human, wherein each CT scan comprises a plurality of pixels;
means for determining from the plurality of CT scans, a plurality of respiratory features;
means for selecting an optimal respiratory feature from the plurality of respiratory features; and
means for generating a respiratory signal directly from the plurality of CT scans based on the plurality of respiratory features.

29. The system of claim 28, further comprising, for each respiratory feature:
means for receiving CT scans from which the respiratory feature was determined;
means for identifying respiratory signals generated based on the received CT scans; and
means for deriving, from the identified respiratory signals, a spatial coherence which is an average pair-wise correlation coefficient, wherein the correlation coefficient is a measure of a quality of the respiratory feature, wherein the spatial coherence is calculated as
$$\frac{1}{{N}^{2}} {\sum }_{i = 1}^{N} {\sum }_{j = 1}^{N} \frac{{\sum }_{t = 1}^{T} ( {s}_{t}^{i} - {\overline{s}}^{i} ) ( {s}_{t}^{j} - {\overline{s}}^{j} )}{\sqrt{{( {s}_{t}^{i} - {\overline{s}}^{i} )}^{2} {( {s}_{t}^{j} - {\overline{s}}^{j} )}^{2}}} ,$$
wherein sⁱₜ is the respiratory signal at the ith slice position and at a particular couch position, N is a number of slice positions per couch position, T is a number of reconstructed axial CT slices per slice location, and
$${\overline{s}}^{i} = \frac{1}{T} {\sum }_{t = 1}^{T} {s}_{t}^{i}$$
is an average of sⁱₜ over time.
